(19) United States
(12) Patent Application Publication
(10) Pub. No.: US 2012/0093720 A1
(43) Pub. Date: Apr. 19, 2012

(54) Molecularly Imprinted Polymers for Use as Imaging and Therapeutics Agents

(76) Inventors: Robert Jones, Columbia, MD (US); Thomas Beck, Catonsville, MD (US)

(21) Appl. No.: 13/274,093

(22) Filed: Oct. 14, 2011

Related U.S. Application Data
(60) Provisional application No. 61/393,881, filed on Oct. 16, 2010.

(51) Int. Cl. A61K 49/00; A61K 51/06; C08G 65/34

(52) U.S. Cl. 424/1.65; 528/425; 528/422; 528/271; 424/9.1

(57) ABSTRACT

The invention described herein provides biocompatible molecularly imprinted polymers (MIPs) that are non-toxic, water soluble, small molecular weight, and degradable in a living body. The MIPs are capable of binding to all or a portion of a specific target macromolecule associated with a disease located within the living body and they are derivatized for stealth for in vivo applications to avoid the reticuloendothelial system. The MIPs of the invention are functionalized to an amine or carboxyl group and are derivatized with an imaging and/or therapeutic agent for taking images of the disease and/or for providing therapy. The macromolecule can be selected from a group consisting of proteins, glycoproteins, lipoproteins, peptidoglycans, peptides, polypeptides, polynucleotides, and polysaccharides. The invention described herein also provides methods and kits wherein MIPs of the invention can be employed as targeted imaging and therapeutic agents.

### FIELD OF THE INVENTION

This patent application claims priority from U.S. Provisional Patent Application Ser. No. 61/393,881, which was filed on Oct. 16, 2010.

The invention described herein generally relates to biocompatible molecularly imprinted polymers (MIPs) that can be employed as targeted imaging agents and therapeutics in vivo, including methods of making and using such MIPs.

### BACKGROUND OF THE INVENTION

Molecular Imaging emerged in the early twenty-first century as a discipline at the intersection of molecular biology and in vivo imaging. It enables the visualization of the cellular function and the follow-up of the molecular process in living organisms without perturbing them. The multiple and numerous potentialities of this field are applicable to the diagnosis of diseases such as cancer, imaging of internal infections, and neurological and cardiovascular diseases. This technique also contributes to improving the treatment of these disorders by optimizing the pre-clinical and clinical tests of new medication. They are also expected to have a major economic impact due to earlier and more precise diagnosis.

Molecular imaging differs from traditional imaging in that probes known as biomarkers are used to help image particular targets or pathways. Biomarkers interact chemically with their surroundings and in turn alter the image according to molecular changes occurring within the area of interest. This process is markedly different from previous methods of imaging which primarily imaged differences in qualities such as density or water content. This ability to image fine molecular changes opens up an incredible number of exciting possibilities for medical application, including early detection and treatment of disease and basic pharmaceutical development. Furthermore, molecular imaging allows for quantitative tests, imparting a greater degree of objectivity to the study of these areas.

Many areas of research are being conducted in the field of molecular imaging. Much research is currently centered on detecting what is known as a predisease state or molecular states that occur before typical symptoms of a disease are detected. Other important veins of research are the imaging of gene expression and the development of novel biomarkers. The invention described herein utilizes Molecularly Imprinted Polymers (MIPs) in the technology of molecular imaging.

MIPs are engineered cross-linked polymers that exhibit high affinity and selectivity towards a single compound or a family of related compounds. MIPs are able to bind analytes even when these are present in complex matrices (e.g., plasma, urine, muscle tissue, food matrices, environmental samples, process solutions etc). An important strength of MIPs is that they are able to bind to trace levels of target molecule, in the presence of large excess of other compounds that have similar physico-chemical properties. Unlike most separation particles that exhibit only non-selective interactions, MIPs have a selective synthetic recognition site (or imprint), which is sterically and chemically complementary to a particular target or class of targets. MIPs are economical and fast to produce and are robust and stable under storage. They can be used at elevated temperatures, in organic solvents and at extreme pH values. They also display a higher sample load capacity than is typical for immunoaffinity based sorbents. This results in higher recoveries for analytical applications and suitability of using the sorbents for semi-preparative or preparative scale separations.

Molecular imprinting involves arranging polymerizable functional monomers around a template (for example, a pseudo-target molecule, an analog of the target molecule, all or portion of the actual target molecule, etc., followed by polymerization and template removal. The arrangement is typically achieved by: (i) non-covalent interactions (e. g., H-bonds, ion pair interactions) or (ii) reversible covalent interactions. After template removal, these molecularly imprinted polymers can recognize and bind to the actual target molecule.

MIPs hold several advantages over antibodies for diagnostics and sample analysis, due to their controlled synthesis and remarkable stability. Molecular imprinting originates from the concept of creating tailor-made recognition sites in polymers by template polymerization (Mosbach K. et al., Bio/Technology, 1996, 14, 163-170; Ansell R. J. et al., Curr. Opin. Biotechnol., 1996, 7, 89-94; Wulff G. Angew. Chem. Int. Ed. Engl., 1995, 34, 1812-32; Vidyasankar S. et al., Curr. Opin. Biotechnol., 1995, 6, 218-224; and Shea K. J, Trends In Polymer Science, 1994, 2, 166-173). Molecularly imprinted polymers demonstrated remarkable recognition properties that were applied in various fields such as drug separation (Fischer L., et al., J. Am. Chem. Soc, 1991, 113, 9358-9360; Kempe M, et al., J. Chromatogr., 1994, 664, 276-279; Nilsson K., et al., J. Chromatogr., 1994, 680, 57-61), receptor mimics (Ramstrom O., et al., Tetrahedron: Asymmetry, 1994, 5, 649-656; Ramstrom O., et al., J. MoI. Recogn., 1996, 9, 691-696; Andersson L. L, et al., Proc. Natl. Acad. Sci., 1995, 92, 4788-4792; Andersson L. L, Anal. Chem., 1996, 68, 111-117), bio-mimetic sensors (Kriz D., et al., Anal. Chem., 1995, 67, 2142-2144), antibody mimics (Vlatakis G., et al., Nature, 1993, 361, 645- 647), template-assisted synthesis (Bystrom S. E., et al, J. Am. Chem. Soc, 1993, 115, 2081-2083), and catalysis (Muller R., et al., Makromol. Chem., 1993, 14, 637-641; Beach J. V., et al., J. Am. Chem. Soc, 1994, Vol. 116, 379-380).

The great potential embodied in MIPs resulted in numerous inventions for analytical devices and methods of detection of various targets, based on molecular imprinting, reviewed by Ye and Haupt (Anal. Bioanal. Chem. 2004, 378, 1887-1897). Some examples of MIP-based sensors are described in U.S. Pat. Nos. 5,587,273, 6,680,210, 6,833,274, 6,967,103 and 6,461,873. Using MIPs combined with displacement of analyte-marker conjugate was shown to be practical in several laboratories (Vlatakis G. et al., Nature, 1993, 361, 645-647, Levi et al., 1997, Anal. Chem. 69. 2017-2021; Nathaniel T. et al., J. Am. Chem. Soc. 2005, 127, 5695-5700; Nicholls C. et al, Biosens. Bioelec, 2006, 21, 1171-1177).

To date, molecular imprints have had limited application to the binding of larger molecules including macromolecules. Synthetic polymers which selectively bind amino acid derivatives and peptides were created using the target amino acid derivative or peptide as a template (Kemp, 1996, Anal. Chem. 68:1948-1953). Imprints have also been created which bind to nucleotide derivatives (Spivak and Shea, 1998, Macromolecules 31:2160-2165). Ionic molecular images of polypeptides have been created by mixing a matrix material with the intact polypeptide chain to be bound by the molecular image (U.S. Pat. No. 5,756,717). Molecular imprints of cytochrome c, hemoglobin and myoglobin, respectively, have been prepared by polymerizing acrylamide in the presence of each intact protein. An imprint of horse myoglobin selectively bound horse myoglobin from a mixture of proteins including whale myoglobin (U.S. Pat. No. 5,814,223).

Although the methods of molecular imprinting have shown limited success at selectively binding macromolecules, the methods have not been utilized in molecular imaging. These shortcomings in the art are overcome by the invention described below, which in one aspect provides MIPs useful in targeted molecular imaging for detecting, identifying and treating a disease state. Generally, the imprint compositions of the invention described below comprise a matrix material defining an imprint of all or portion of macromolecule associated with a disease state. In one embodiment of the invention, MIPs of the invention may be conjugated to a radionuclide, and transported to the target organ in the body to be imaged. Potential advantages of MIP-based materials include: specificity comparable to a biorecognition element; robustness and stability under extreme chemical and physical conditions; and an ability to design recognition sites for target molecules that lack suitable biorecognition elements.

### SUMMARY OF THE INVENTION

One embodiment of the invention provides a biocompatible molecularly imprinted polymer (MIP) wherein the MIP is derivatized for stealth for in vivo applications to avoid the reticuloendothelial system, wherein the MIP is capable of binding to all or a portion of a specific target macromolecule located in a living body.

In one embodiment of the invention wherein said stealth is achieved by covalently attaching polyethylene glycol polymer or derivatives of polyethylene glycol to the MIP by the PEGylation process.

In one embodiment of the invention the MIP is functionalized to an amine or carboxyl group and is derivatized for imaging using a variety of imaging agents.

In one embodiment of the invention the MIP is functionalized to an amine or carboxyl group and is derivatized for a therapeutic agent in conjunction with or in lieu of an imaging agent.

In one embodiment of the invention the MIP is conjugated to said imaging and/or therapeutic agents.

In one embodiment of the invention said macromolecule is immunogenic.

In one embodiment of the invention said macromolecule is non-immunogenic.

In one embodiment of the invention said macromolecule is a cancer antigen.

In one embodiment of the invention said cancer antigen is expressed by a malignant solid tumor.

In one embodiment of the invention said cancer antigen is expressed by a malignant hematopoietic cell.

In one embodiment of the invention said macromolecule is an integral part of a microorganism.

In one embodiment of the invention said microorganism is the cause of an infection in vivo.

In one embodiment of the invention said macromolecules are selected from a group consisting of proteins, glycoproteins, lipoproteins, peptidoglycans, peptides, polypeptides, polynucleotides, and polysaccharides.

In one embodiment of the invention said imaging agent is selected form a group consisting of radionuclides, fluorophores, metalloproteins, iodine and metals such as gadolinium, gold and platinum.

In one embodiment of the invention said therapeutic agent is selected from a group consisting of radionuclides, toxins, and metals such as gold, silver and platinum.

In one embodiment of the invention said fluorophore emits radiation in the near-infrared region of the spectrum for tissue penetration.

In one embodiment of the invention the MIP/imaging agent and/or therapeutic agent conjugation is injected, ingested, or inhaled into a living body.

In one embodiment of the invention a camera located outside said living body is capable of taking images using radiation emitted by said imaging agent.

### DETAILED DESCRIPTION OF THE INVENTION

Imaging Modalities

There are many different modalities that can be used for noninvasive molecular imaging. Examples include:

Magnetic Resonance Imaging (MRI)

MRI has the advantages of having very high spatial resolution and is very adept at morphological imaging and functional imaging. MRI does have several disadvantages though. First, MRI has a sensitivity of around 10⁻³ mol/L to 10⁻⁵ mol/L which compared to other types of imaging can be very limiting. This problem stems from the fact that the difference between atoms in the high energy state and the low energy state is very small. For example, at 1.5 teslas the difference between high and low energy states is approximately 9 molecules per 2 million. Improvements to increase MRI sensitivity include hyperpolarization by increasing magnetic field strength, optical pumping, or dynamic nuclear polarization. There are also a variety of signal amplification schemes based on chemical exchange that increase sensitivity.

Optical Imaging

There are a number of approaches used for optical imaging. The various methods depend upon fluorescence, bioluminescence, absorption or reflectance as the source of contrast. The most valuable attribute of Optical imaging, similar to ultrasound, is that it does not have strong safety concerns like the other medical imaging modalities.

The downside of optical imaging is the lack of penetration depth, especially when working at visible wavelengths. Depth of penetration is related to the absorption and scattering of light, which is primarily a function of the wavelength of the excitation source. Light is absorbed by endogenous chromophores found in living tissue (e.g. hemoglobin, melanin, and lipids). In general, light absorption and scattering decreases with increasing wavelength. Below ˜700 nm (e.g. visible wavelengths), these effects result in shallow penetration depths of only a few millimeters. Thus, in the visible region of the spectrum, only superficial assessment of tissue features is possible. Above 900 nm, water absorption can interfere with signal-to-background ratio. Because the absorption coefficient of tissue is considerably lower in the near infrared (NIR) region (700-900 nm), light can penetrate more deeply, to depths of several centimeters.

Fluorescent probes and labels are an important tool for optical imaging. A number of near-infrared (NIR) fluorophores have been employed for in vivo imaging, including Kodak X-SIGHT Dyes and Conjugates, DyLight 750 and 800 Fluors, Cy 5.5 and 7 Fluors, Alexa Fluor 680 and 750 Dyes, IRDye 680 and 800CW Fluors. Quantum dots, with their photostability and bright emissions, have generated a great deal of interest; however, their size precludes efficient clearance from the circulatory and renal systems while exhibiting long-term toxicity.

Several studies have demonstrated the use of infrared dye-labeled probes in optical imaging. (1) In a comparison of gamma scintigraphy and NIR imaging, a cyclopentapeptide dual-labeled with 111 indium and an NIR fluorophore was used to image αvβ3-integrin positive melanoma xenografts. (2) Near-infrared labeled RGD targeting αvβ3-integrin has been used in numerous studies to target a variety of cancers. (3) An NIR fluorophore has been conjugated to epidermal growth factor (EGF) for imaging of tumor progression. (4) An NIR fluorophore was compared to Cy5.5, suggesting that longer-wavelength dyes may produce more effective targeting agents for optical imaging. (5) Pamidronate has been labeled with an NIR fluorophore and used as a bone imaging agent to detect osteoblastic activity in a living animal. (6) An NIR fluorophore-labeled GPI, a potent inhibitor of PSMA (prostate specific membrane antigen). (7) Use of human serum albumin labeled with an NIR fluorophore as a tracking agent for mapping of sentinel lymph nodes. (8) 2-Deoxy-D-glucose labeled with an NIR fluorophore.

Single Photon Emission Computed Tomography (SPECT)

The main purpose of SPECT when used in brain imaging is to measure the regional cerebral blood flow (rCBF). The development of computed tomography in the 1970s allowed mapping of the distribution of the radioisotopes in the brain, and led to the technique now called SPECT.

The imaging agent used in SPECT emits gamma rays, as opposed to the positron emitters (such as ¹⁸F) used in PET. There are a range of radiotracers (such as ^{99m}Tc, ¹¹¹In, ¹²³I, ²⁰¹Tl) that can be used, depending on the specific application.

Xenon (¹³³Xe) gas is one such radiotracer. It has been shown to be valuable for diagnostic inhalation studies for the evaluation of pulmonary function; for imaging the lungs; and may also be used to assess rCBF. Detection of this gas occurs via a gamma camera—which is a scintillation detector consisting of a collimator, a NaI crystal, and a set of photomultiplier tubes.

By rotating the gamma camera around the head, a three dimensional image of the distribution of the radiotracer can be obtained by employing filtered back projection or other tomographic techniques. The radioisotopes used in SPECT have relatively long half lives (a few hours to a few days) making them easy to produce and relatively cheap. This represents the major advantage of SPECT as a brain imaging technique, since it is significantly cheaper than either PET or fMRI. However it lacks good spatial (i.e., where exactly the particle is) or temporal (i.e., did the contrast agent signal happen at this millisecond, or that millisecond) resolution. Additionally, due to the radioactivity of the contrast agent, there are safety aspects concerning the administration of radioisotopes to the subject, especially for serial studies.

Positron Emission Tomography (PET)

Positron emission tomography is a nuclear medicine imaging technique which produces a three-dimensional image or picture of functional processes in the body. The theory behind PET is simple enough. First a molecule is tagged with a positron emitting isotope. These positrons annihilate with nearby electrons, emitting two 511,000 eV photons, directed 180 degrees apart in opposite directions. These photons are then detected by the scanner which can estimate the density of positron annihilations in a specific area. When enough interactions and annihilations have occurred, the density of the original molecule may be measured in that area. Typical isotopes include ¹¹C, ¹³N, ¹⁵O, ¹⁸F, ⁶⁴Cu, ⁶²Cu, ¹²⁴I, ⁷⁶Br, ⁸²Rb and ⁶⁸Ga, with ¹⁸F being the most clinically utilized. One of the major disadvantages of PET is that most of the probes must be made with a cyclotron. Most of these probes also have a half life measured in hours, forcing the cyclotron to be on site. These factors can make PET prohibitively expensive. PET imaging does have many advantages though. First and foremost is its sensitivity: a typical PET scanner can detect between 10⁻¹¹ mol/L to 10⁻¹² mol/L concentrations.

Ultrasound

Medical sonography (ultrasonography) is an ultrasound-based diagnostic medical imaging technique used to visualize muscles, tendons, and many internal organs, to capture their size, structure and any pathological lesions with real time tomographic images. Ultrasound has been used by sonographers to image the human body for at least 50 years and has become one of the most widely used diagnostic tools in modern medicine. The technology is relatively inexpensive and portable, especially when compared with other techniques, such as magnetic resonance imaging (MRI) and computed tomography (CT). Ultrasound is also used to visualize fetuses during routine and emergency prenatal care. Such diagnostic applications used during pregnancy are referred to as obstetric sonography.

Nuclear Medicine

Nuclear medicine is a branch or specialty of medicine and medical imaging that uses radioactive isotopes (radionuclides) and relies on the process of radioactive decay in the diagnosis and treatment of disease. In nuclear medicine procedures, radionuclides are combined with other chemical compounds or pharmaceuticals to form radiopharmaceuticals. These radiopharmaceuticals, once administered to the patient, can localize to specific organs or cellular receptors. This unique ability of radiopharmaceticals allow nuclear medicine to diagnose or treat a disease based on the cellular function and physiology rather than relying on the anatomy.

In nuclear medicine imaging, radiopharmaceuticals are taken internally, for example intravenously or orally. Then, external detectors (gamma cameras) capture and form images from the radiation emitted by the radiopharmaceuticals. This process is unlike a diagnostic X-ray where external radiation is passed through the body to form an image. Nuclear medicine imaging may also be referred to as radionuclide imaging or nuclear scintigraphy.

Nuclear medicine tests differ from most other imaging modalities in that diagnostic tests primarily show the physiological function of the system being investigated as opposed to traditional anatomical imaging such as CT or MRI. Nuclear Medicine imaging studies are generally more organ or tissue specific (e.g.: lungs scan, heart scan, bone scan, brain scan, etc.) than those in conventional radiology imaging, which focus on a particular section of the body (e.g.: chest X-ray, abdomen/pelvis CT scan, head CT scan, etc.).

Diagnostic tests in nuclear medicine exploit the way that the body handles substances differently when there is disease or pathology present. The radionuclide introduced into the body is often chemically bound to a complex that acts characteristically within the body; this is commonly known as a tracer. In the presence of disease, a tracer will often be distributed around the body and/or processed differently. For example, the ligand methylene-diphosphonate (MDP) can be preferentially taken up by bone. By chemically attaching technetium-99m to MDP, radioactivity can be transported and attached to bone via the hydroxyapatite for imaging. Any increased physiological function, such as due to a fracture in the bone, will usually mean increased concentration of the tracer. This often results in the appearance of a ‘hot-spot’ which is a focal increase in radio-accumulation, or a general increase in radio-accumulation throughout the physiological system. Some disease processes result in the exclusion of a tracer, resulting in the appearance of a ‘cold-spot’. Many tracer complexes have been developed in order to image or treat many different organs, glands, and physiological processes.

Molecularly Imprinted Polymers (MIPs) as Radiopharmaceuticals

With the possible exception of Ultrasound, MIPs can be employed as an imaging agent in lieu of antibodies in all the imaging modalities discussed above. MIPs can serve as targeted imaging agents in vivo. MIPs possess a number of qualities tractable for the development of imaging agents, the most important of which is the ability to be generated toward virtually any molecular target. The MIPs can be functionalized for targeting and imaging in ways akin to what is currently being performed with respect to nanoparticles.

MIPs can serve as radiopharmaceuticals. A MIPs ligand can be developed that is specific for proteins of the target organ such as the prostate, brain, myocardium, thyroid, lungs, liver, gallbladder, kidneys, skeleton, blood and tumors. A radionuclide, such as technetium-99m, can be conjugated to the MIPs ligand and the radionuclide can be transported to the target organ in the body to be imaged.

Method of Making MIPs:

The MIPs of the invention can be prepared in accordance with any technique known to those skilled in the art using all or portion of a macromolecule, associated with diagnosing or treating a disease state or cancer, as a template molecule. These methods include covalent imprinting (Wulff, 1982, Pure & Appl. Chem., 54, 2093-2102) whereby the monomers are covalently attached to the template and polymerized using a cross-linker. Subsequently, the template is cleaved from the polymer leaving template-specific binding cavities. Alternatively, a non-covalent imprinting method such as disclosed by U.S. Pat. No. 5,110,833, which portion is specifically incorporated herein by reference, may be used, whereby the monomers interact with the target molecule by non-covalent forces, and are then connected via a cross-linker to form target specific binding sites after removal of the target molecule. Combinations and variations on these methods may be used to construct thin molecularly imprinted films and membranes (Hong et al., 1998 Chem. Mater., 10, 1029-1033); imprinting on the surface of solid supports (Blanco-López, et. al., 2004, Anal. Bioanal. Chem., 378, 1922-1928; Sulitzky C. et al., 2002 Macromolecules, 35, 79-91); and microspheres (Ye et al., 2000, Macromolecules, 33, 8239-8245). Further, methods for preparing MIPs are described in U.S. Pat. Nos. 4,406,792, 4,415,655, 4,532,232, 4,935,365, 4,960,762, 5,015,576, 5,208,155, 5,310,648, 5,321,102, 5,372,719, 5,786,428, 6,063,637, and 6,593,142, the portions of all of which disclosing the methods for preparing MIPs are specifically incorporated herein by reference.

Generally, molecular imprinting involves making a polymer cast of a template molecule, wherein the template includes, but is not limited to, an epitope of a protein, glycoprotein, lipoprotein, peptide, polypeptide, peptidoglycans, polysaccharides or a nucleotide sequence. An epitope is, in accordance to the invention, a portion of a molecule that is specifically recognized by the MIP. Accordingly, the MIP can be a known immune reaction triggering chemical sequence part of a molecule or it can be a part of a molecule not typically considered as causing an immune effect. The process of making the polymer cast involves dissolving the template molecule to be imprinted in a suitable solvent. Normally, an imprint composition comprising a co-monomer, cross-linking monomer and a polymerization initiator is added to the solvent comprising the desired template. Radiation (photochemical or ionizing) or thermal energy is then applied to the reaction mixture, comprising the imprint composition and the template, to drive the polymerization process, ultimately resulting in the formation of a solid polymer. The resulting polymer may be processed using conventional polymer processing technologies, assuming those processes do not alter the structure of the molecularly imprinted sites. The imprinted molecule is extracted using methods appropriate for dissociating the template molecule from the polymer. Details of template molecule dissociation from the polymer are dependent upon the nature of the chemical interaction between the target molecule and the polymer binding site. The polymer dissociated from the template molecule possesses binding sites optimized for the structural and electronic properties of such template molecule.

For example, MIPs in accordance with one aspect of the present invention can be prepared by (A) providing the reaction product of a polymerizable porphyrin derivative and a template molecule; (B) copolymerizing the reaction product of step (A) with monomer and crosslinking agent to form a polymer; and (C) removing the template molecule from the polymer to provide a molecularly imprinted polymer which exhibits selective binding affinity for the template molecule and undergoes a detectable change in absorption and/or emission of electromagnetic radiation when the target molecule binds thereto. The polymerization reaction mixture for preparation of MIP therefore constitutes the reaction product of step (A), one or more polymerizable monomers, an effective amount of one or more crosslinking agents to impart a sufficiently rigid structure to the polymer end-product, inert solvent, and a free radical or other appropriate initiator. Mixtures of monomers and crosslinking agents can be used in the polymerization method. The amounts of polymerizable porphyrin, monomer and crosslinking agents can vary broadly, depending on the specific nature/reactivities of the polymerizable porphyrin, monomer and crosslinking agent chosen as well as the specific sensor application and environment in which the polymer/sensor will be ultimately employed. The relative amounts of each reactant can be varied to achieve desired concentrations of porphyrin in the polymer support structure. The solvent, temperature and means of polymerization can be varied in order to obtain polymeric materials of optimal physical or chemical features, for example, porosity, stability, and hydrophilicity. The solvent can also be chosen based on its ability to solubilize all the various components of the reaction mixture.

According to one embodiment of the invention described herein, the MIPs comprise lanthanide-containing polymeric structures that exhibit selective binding characteristics towards a target. The polymerization step comprises co-polymerizing a chelated lanthanide-template complex with one or more cross-linking monomers, and optionally, one or more additional matrix monomers to form a polymer structure. Any of a wide range of lanthanide metal salts capable of dissociating in solution to form a lanthanide ion, and combinations of two or more thereof, are suitable for use in the invention described herein. Examples of suitable lanthanide salts include, but are not limited to, halides, nitrates, perchlorates, and the like, of lanthanum (La), cerium (Ce), praseodymium (Pr), neodymium (Nd), promethium (Pm), samarium (Sm), europium (Eu), gadolinium (Gd), terbium (Th), dysprosium (Dy), holmium (Ho), erbium (Er), thulium (Tm), ytterbium (Yb), and lutetium (Lu). MIPs can be used as part of an optical sensor device to selectively capture the target, by associating such molecules with MIP lanthanide binding sites. Such MIPs act as a source of luminescence, which can be analyzed in detecting the presence of a target molecule. Any of a wide range of suitable detectors can be used according to the invention described herein. Non-limiting examples of suitable detectors include a spectrophotometer, spectrometer (gas or mass), photomultiplier tube, monochromator equipped with a CCD camera, filters, the naked eye, combinations of two or more thereof, and the like.

The invention described herein additionally presents a method for forming a reliable chemical sensor platform based on site selectively tagged and templated molecularly imprinted polymers (SSTT-MIP). The SSTT-MIP strategy used in the present method provides a way to form a MIP having a templated site specific for an analyte and at which a reporter molecule can also be attached. In this way, analyte detection can be carried out with a higher efficiency in comparison to methodologies without any provision for such positioning. With this invention, measurement characteristics such as signal-to-background and signal-to-noise ratios are expected to be improved.

The invention also provides molecularly imprinted polymer platforms in which templated sites are formed for specific target molecules. In one embodiment, the polymer platforms can be provided wherein the templated sites have at least one reporter molecule bonded to a reactive group at the site. In one embodiment, the polymer platform comprises xerogels or aerogels. Methods to develop sensors for the detection of a wide variety of targets are described by Bright et al., in U.S. Publication No. 2005/0227258, the portions of which that describe development of templated sites for specific target molecules, is incorporated herein by reference.

For example, the template is chosen such that it forms bonds with the polymer platform in excess of the number of those which will be formed by the bound target. The template can have at least one additional reactive group so as to be able to bind to the polymer matrix at an additional site relative to an intended target. In one embodiment, the number of reactive groups on the template which link it to the polymer platform, is at least one more than the number of reactive groups on the corresponding target. The removal of the template results in cavities within the polymer platform. Exposed at each templated site are reactive groups which are responsible for target recognition. However, as a consequence of the additional reactive group(s) mentioned above, when the template is cleaved from the cavity, the cavity bears one or more reactive groups in excess of the groups needed to bind the target. The extra group(s) is (are) used to bond with reporter molecules. Once the reporter molecule(s) is (are) bound at the templated site, the absorbance/luminescence from the target bound MIP can be measured and a change in UV, visible or IR absorbance/luminescence properties of the reporter (e.g., absorbance spectra, excitation and emission spectra, excited-state luminescence lifetime and/or luminescence polarization) indicates the presence of target at the templated site. The total change in absorbance/luminescence is generally proportional to the concentration of target molecule in the sample.

Preferably, the conditions under which the template molecule is imprinted are similar or identical to the conditions under which the macromolecule is to be captured. For instance, if the macromolecule is to be captured under denaturing conditions, then the template molecule should be imprinted under the same denaturing conditions. Similarly, if the macromolecule is to be captured under native conditions, then the template molecule should be imprinted under the same native conditions. Native and denaturing conditions are well-known to those of skill in the art. Many heat-sensitive compounds that can be used to make imprint compositions according to the invention are known in the art and include, by way of example and not limitation, hydrogels such as agarose, gelatins, moldable plastics, etc. Examples of other suitable hydrogels are described in U.S. Pat. No. 6,018,033, U.S. Pat. No. 5,277,915, U.S. Pat. No. 4,024,073, and U.S. Pat. No. 4,452,892, the portions of all of which that relate to imprinting are incorporated herein by reference.

Suitable non-limiting examples of monomers that can be used for preparing a polymer of the present invention include methylmethacrylate, other alkyl methacrylates, alkylacrylates, ally or aryl acrylates and methacrylates, cyanoacrylate, styrene, α-methyl styrene, vinyl esters, including vinyl acetate, vinyl chloride, methyl vinyl ketone, vinylidene chloride, acrylamide, methacrylamide, acrylonitrile, methacrylonitrile, ethylene glycol diacrylate, pentaerythritol dimethacrylate, pentaerythritol diacrylate, N,N′-methylenebisacrylamide, N,N′-ethylenebisacrylamide and N,N′-(1,2-dihydroxyethylene)bisacrylamide. Depending upon the choice of the monomers used, the polymer particles will have a variety of physical and mechanical properties, such as hydrophobicity/hydrophilicity, mechanical strength and ease or resistance to swelling in the presence of solvents.

The MIPs of the invention may take a variety of different forms. For example, they may be in the form of individual beads, disks, ellipses, or other regular or irregular shapes (collectively referred to as “beads”), or in the form of sheets. Each bead or sheet may comprise imprint cavities of a single template molecule, or they may comprise imprint cavities of two or more same or different template molecules. In one embodiment, the MIPs comprise imprint cavities of a plurality of different template molecules arranged in an array or other pattern such that the relative positions of the imprint cavities within the array or pattern correlate with their identities, i.e. the identities of the template molecules used to create them. Each position or address within the array may comprise an imprint cavity of a single template molecule, or imprint cavities of a plurality of different template molecules, depending upon the application. Moreover, the entire array or pattern may comprise unique imprint cavities, or may include redundancies, depending upon the application.

In one embodiment, the invention provides methods of manufacturing matrix materials comprising the imprint compositions. Such matrix materials include, but are not limited to, substances that are capable of undergoing a physical change from a fluid state to a semi-solid or solid state. In the fluid state, the particles of a matrix material move easily among themselves, and the material retains little or no definite form. A matrix material in the fluid state can be mixed with other compounds, including template molecules. In the semi-solid or solid state, the matrix materials are capable of forming and retaining cavities that complement the shape of template molecules. Examples of such matrix materials include heat sensitive hydrogels such as agarose, polymers such as acrylamide, and cross-linked polymers.

The MIP of the invention is biocompatible. By “biocompatible,” it is indicated that the MIP is, generally, non-toxic, soluble in a physiological solution, of a molecular weight compatible with its circulation in a given tissue system, not causing an adverse reaction such as an immune reaction, and having a desirable chemical and biological half-life.

In one embodiment, the MIP of the invention is “non-toxic.” Non toxicity is a practical, functional aspect, measured during in vitro-cellular-testing or in vivo, in a model animal or a human patient. Non-toxicity is not equivalent to absolute lack of morphological or metabolic undesirable effect. Small effects that are not life-threatening and are considered by an artisan skilled in the art as acceptable relative to the advantages provided by use of the MIP of the invention are acceptable and the MIP causing such effect is within the scope of the invention. For example, in certain preferred embodiments, a temporary (less than one week) change in the division time of a cell of about 2% to about 15% would not necessarily qualify as a toxic effect; a temporary change in the respiration rate (up to about 15% of normal rate) would also not necessarily qualify as a toxic effect; temporary changes in appetite or body weight may also be acceptable.

In accordance to one embodiment, the MIP of the invention is “water soluble.” This means that the MIP is soluble in a water or a water-based solution, such as a physiological buffer and, more preferably, in a buffer compatible with delivery of the MIP to a cell or an animal. In a preferred embodiment, the water soluble MIP of the invention, when prepared at a concentration equivalent to its concentration upon delivery to a cell culture or an animal will, upon standard physiological conditions (e.g. temperature) not precipitate/sediment significantly. Significant precipitation is a precipitation out of solution greater than 20% within 24 hrs. (This definition of precipitation does not refer to precipitation in the presence of the targeted molecule.) Preferably, the precipitation is yet more reduced and at least about 85%, 90%, 95% or more of the MIP stays in solution under the conditions described in this paragraph. In accordance to a further preferred embodiment, the MIP core comprises components including methacrylate and methacrylamide. In a still more preferred embodiment, the MIP comprises about 15 mol % of allyl methacrylate and 8 mol % N-(2-hydroxypropyl) methacrylamide (HPMA).

In accordance to another embodiment, the MIP of the invention is a small molecule. Preferably it is of a size and shape allowing its distribution at least through the cardiovascular system, although its size is determined in part by its intended function and functional space in vivo. Preferably, the MIP is no larger than about 35 kD, more preferably less than about 30 kD, 25 kD, 20 kD, 15 kD, 10 kD, or 5 kD. More preferably, the MIP is between about 10 kD and 19 kD. Yet more preferably, the preferred sizes listed in this paragraph refer to the MIP made more “stealthy” by the addition of a masking component, such as by glycosylation.

In accordance to another embodiment, the MIP of the invention is created to have increased resistance to biological rejection, or an increased biological half-life for any reason, by various modifications to the MIP. A preferred modification is glycosylation. For example, PEG groups are attached, preferably covalently attached. More preferably, the PEG group(s) attached have small molecular weights. The MIP of the invention can be made from a variety of polymers and in different designs. This allows designing the MIP with control of its chemical and biological half-life in mind. Clearly, storage would suggest advantages for a long chemical half-life. A MIP derivative intended as a therapeutic agent might benefit from a long biological half-life. However, a MIP derivative intended as a diagnostic/imaging agent could benefit from a shorter half-life. However, practically speaking, even a MIP derivative used as an imaging agent is likely desired to have a biological half-life of at least about 30 minutes and, more preferably, to have a biological half-life of at least about 1 hr., 2 hrs., 12 hrs., 24 hrs., or longer.

In accordance to another embodiment, the MIP of the invention is selected for and will be recognized by a skilled artisan to have sufficiently high binding affinity (selectivity) and specificity to insure that the MIP is highly effective in picking up the target molecule, even at low concentrations of the target molecule.

The template molecule of the invention described herein can be selected from the group consisting of all or a portion of a macromolecule associated with a host cell, host cell, or host cell factors, and analogs thereof. The portion to which the template molecule corresponds may be an internal portion of the macromolecule and/or an external portion, and/or a terminal portion of the macromolecule. Alternatively, the portion may be a side-group or modification of the macromolecule, such as a polysaccharide group of a glycoprotein macromolecule, or a portion thereof. The template molecule can be selected from the group consisting of proteins, enzymes, antibodies, antigens, hormones, peptides, polynucleotides, polynucleic acids, polypeptides, steroids, polyfatty acids, polyglucotides, polyglycerides, lipids, polysaccharides, whole cells, pathogens, viruses, triglycerides, nucleotides, nucleic acid bases and their conjugates, byproducts of biosynthesis of biomolecules, and combinations thereof.

For example, the template molecule can be designed to comprise the molecular structure of all or portion of a macromolecules associated with target organs such as prostate, brain, myocardium, thyroid, lungs, liver, gallbladder, kidneys, and tumors thereof. The template molecule can be designed to comprise the molecular structure of all or portion of a macromolecule associated with a host cell, such as cell surface receptors. Non-limiting examples of host cells include melanocytes, keratinocytes, fibroblasts, endothelial cells, epithelial cells, and dendritic cells. Additionally, the template molecules can be designed to comprise the molecular structure of all or portion of a host cell factors, such as cytokines.

Macromolecules associated with prostate, brain, myocardium, thyroid, lungs, liver, gallbladder, kidneys, and tumors thereof that can be used to diagnose, treat or prevent using the imprint compositions of the invention include any type of macromolecule from which a template molecule can be designed and constructed according to the principles taught herein. Non-limiting examples of the macromolecules include polysaccharides, proteins, glycoproteins, peptidoglycans, lipoproteins, peptides, polypeptides, and polynucleotides, and other macromolecular targets that will be apparent to those of skill in the art.

In general, the structural units of the macromolecule to which the template molecule corresponds are contiguous within the primary structure of the macromolecule. If one of skill in the art can identify a terminus or termini in the primary structure of the macromolecule, then a preferred template molecule corresponds to a template that includes a terminus of the macromolecule. Alternatively, the portion of the macromolecule to which the template molecule corresponds can be expressed in size as a fraction of the size of the entire macromolecule. For example, template molecules can correspond to a portion of the macromolecule that consists of from 1% to 5%, from 1 to 10%, from 1 to 15%, from 1 to 20%, from 1 to 25%, from 1 to 30%, from 1 to 35%, from 1 to 40%, from 1 to 50%, from 1 to 60%, from 1 to 70%, from 1 to 80%, from 1 to 90%, from 1 to 95%, or from 1 to 99% of the structure of the entire macromolecule. Preferably, template molecules have a primary structure that corresponds to a contiguous portion of the primary structure of the macromolecule.

If the macromolecule is a polypeptide, the template molecule can correspond to a portion of the polypeptide that consists of a sequence of amino acids selected from the primary sequence of the polypeptide or an analog thereof. For instance, the portion of the polypeptide can consist of a range of amino acids from the primary structure of the polypeptide consisting of from 1 to 50 amino acids, from 2 to 40 amino acids, from 3 to 30 amino acids, from 3 to 15 amino acids, from 3 to 10 amino acids, from 3 to 50 amino acids, from 4 to 10 amino acids, from 4 to 9 amino acids, from 4 to 8 amino acids, from 4 to 7 amino acids, or from 5 to 7 amino acids. Preferred portions of the macromolecule are those that consist of a contiguous sequence of amino acids from the primary structure of the polypeptide.

When the macromolecule is a polynucleotide, the template molecule can be an oligonucleotide having a sequence of nucleotides selected from the primary sequence of the polynucleotide or an analog thereof. If the polynucleotide has n nucleotides, then the selected sequence of nucleotides can have a length from 1 to (n-1) nucleotides. Alternatively, the selected sequence can contain from 1 to 50 nucleotides, 2 to 40 nucleotides, 3 to 30 nucleotides, 3 to 15 nucleotides, 3 to 10 nucleotides, 4 to 10 nucleotides, 4 to 9 nucleotides, 4 to 8 nucleotides, 4 to 7 nucleotides, or 5 to 7 nucleotides. Preferably, the selected sequence is a contiguous sequence of nucleotides from the primary sequence of the polynucleotide.

It will be understood that as used herein, the expression “macromolecule” is not intended to place specific size limitations upon the molecules that may be identified with the MIPs of the methods described herein. Rather, macromolecules include molecules that comprise a plurality of structural moieties or analogs thereof such that a template molecule corresponding to at least one of the structural moieties can be used to prepare a molecular imprint capable of binding the macromolecule. In one embodiment of the invention, template molecules corresponding to at least two of the structural moieties can be used to prepare a molecular imprint capable of binding the macromolecule. In one embodiment of the invention, template molecules corresponding to at least three of the structural moieties can be used to prepare a molecular imprint capable of binding the macromolecule. In one embodiment of the invention, template molecules corresponding to at least four of the structural moieties can be used to prepare a molecular imprint capable of binding the macromolecule.

By “analog” is meant a molecule that differs from, but is structurally, functionally, and/or chemically related to the reference molecule. The analog may retain the essential properties, functions, or structures of the reference molecule. Most preferably, the analog retains at least one biological function of the reference molecule. Generally, differences are limited so that the structure or sequence of the reference molecule and the analog are similar overall. For example, a peptide analog and its reference peptide may differ in amino acid sequence by one or more substitutions, additions, and/or deletions, in any combination. Other examples of analogs include peptides with minor amino acid variations from the peptides exemplified herein. In particular, peptides containing conservative amino acid replacements, i.e., those that take place within a family of amino acids that are related in their side chains, constitute analogs. A substituted or inserted amino acid residue may or may not be one encoded by the genetic code. An analog of a peptide or polypeptide may be naturally occurring such as an allelic variant, or it may be a variant that is not known to occur naturally. Non-naturally occurring analogs of peptides may be made by direct synthesis, by modification, or by mutagenesis techniques.

The identities of the structural moieties that comprise macromolecules will depend upon the nature of the macromolecule, and may include regions of primary, secondary and/or tertiary structure of the macromolecule. For example, for polypeptide macromolecules the structural moieties may be the individual amino acids composing the polypeptide, or alternatively, if the polypeptide has several structural domains, the structural moieties may be the individual structural domains. For example, a polypeptide may be viewed as being composed of individual amino acids or structural domains as described above and/or saccharide or oligosaccharide structural moieties; a polynucleotide macromolecule may be viewed as being composed of individual nucleotide structural moieties. The macromolecules according to the invention may be derived from virtually any source. They may be obtained from natural sources such as biological samples or from synthetic sources.

MIPs in accordance with one aspect of the present invention can be prepared by (A) providing the reaction product of a polymerizable porphyrin derivative and a template molecule; (B) copolymerizing the reaction product of step (A) with one more monomers and crosslinking agent to form a polymer; and (C) removing the template molecule from the polymer to provide a molecularly imprinted polymer which exhibits selective binding affinity for the template molecule and undergoes a detectable change in absorption and/or emission of electromagnetic radiation when the target molecule binds thereto. The polymerization reaction mixture for preparation of a MIP therefore constitutes the reaction product of step (A), one or more polymerizable monomers, an effective amount of one or more crosslinking agents to impart a sufficiently rigid structure to the polymer end-product, inert solvent, and a free radical or other appropriate initiator. Mixtures of monomers and crosslinking agents can be used in the polymerization method. The amounts of polymerizable porphyrin, monomer and crosslinking agents can vary broadly, depending on the specific nature/reactivities of the polymerizable porphyrin, monomer and crosslinking agent chosen as well as the specific sensor application and environment in which the polymer/sensor will be ultimately employed. The relative amounts of each reactant can be varied to achieve desired concentrations of porphyrin in the polymer support structure. The solvent, temperature and means of polymerization can be varied in order to obtain polymeric materials of optimal physical or chemical features, for example, porosity, stability, and hydrophilicity. The solvent can also be chosen based on its ability to solubilize all the various components of the reaction mixture.

Further, according to another embodiment of the invention described herein, the MIPs may comprise lanthanide-containing polymeric structures that exhibit selective binding characteristics towards a target to be detected by a sensor device or kit of the invention described herein. The polymerization step comprises co-polymerizing a chelated lanthanide-template complex with one or more cross-linking monomers, and optionally, one or more additional matrix monomers to form a polymer structure. Any of a wide range of lanthanide metal salts capable of dissociating in solution to form a lanthanide ion, and combinations of two or more thereof, are suitable for use in the invention described herein. Examples of suitable lanthanide salts include, but are not limited to, halides, nitrates, perchlorates, and the like, of lanthanum (La), cerium (Ce), praseodymium (Pr), neodymium (Nd), promethium (Pm), samarium (Sm), europium (Eu), gadolinium (Gd), terbium (Th), dysprosium (Dy), holmium (Ho), erbium (Er), thulium (Tm), ytterbium (Yb), and lutetium (Lu). MIPs can be used as part of an optical sensor device to selectively capture the target, by associating such molecules with MIP lanthanide binding sites. Such MIPs act as a source of luminescence, which can be analyzed to determine the amount of target in the solution. Any of a wide range of suitable detectors can be used according to the invention described herein. Non-limiting examples of suitable detectors include a spectrophotometer, spectrometer (gas or mass), photomultiplier tube, monochromator equipped with a CCD camera, filters, the naked eye, combinations of two or more thereof, and the like.

The invention described herein additionally presents a method for forming a reliable chemical sensor platform based on site selectively tagged and templated molecularly imprinted polymers (SSTT-MIP). The SSTT-MIP strategy used in the present method provides a way to form a MIP having a templated site specific for an analyte and at which a reporter molecule can also be attached. In this way, analyte detection can be carried out with a higher efficiency in comparison to methodologies without any provision for such positioning. With this invention, measurement characteristics such as signal-to-background and signal-to-noise ratios are expected to be improved.

One embodiment of the invention provides MIPs conjugated to one or more imaging moieties, including but not limited to radiolabels, fluorophores, chromophores, and other well known imaging agents.

One embodiment of the invention provides MIPs comprising one or more diagnostic agents within the core, wherein said diagnostic agents are released upon binding of MIPs to a target molecule. Another aspect of the invention provides MIPs comprising nucleic acid molecules or amino acid molecules having the target molecular structure coupled with one or more diagnostic agents. The diagnostic agents can include, but not limited to, enzymes, radiolabels, fluorophores, chromophores, imaging agents or metal ions.

Examples of radiolabels include, but are not limited to ¹¹⁰In, ¹¹¹In, ¹⁷⁷Lu, ¹⁸F, ⁵²Fe, ⁶²Cu, ⁶⁴Cu, ⁶⁷Cu, ⁶⁷Ga, ⁶⁸Ga, ⁸⁶Y, ⁹⁰Y, ⁸⁹Zr, ^{94m}Tc, ⁹⁴Tc, ^{99m}Tc, ¹²⁰I, ¹²³I, ¹²⁴I, ¹²⁵I, ¹³¹I, ¹⁵⁴⁻¹⁵⁸Gd, ³²P, ¹¹C, ¹³N, ¹⁵O, ¹⁸⁶Re, ¹⁸⁸Re, ⁵¹Mn, ^{52m}Mn, ⁵⁵Co, ⁷²As, ⁷⁵Br, ⁷⁶Br, ^{82m}Rb, ⁸³Sr, or other β-, gamma-, x-ray or positron-emitters. Examples of fluorophores include, but are not limited to, fluorescein isothiocyanate, rhodamine, phycoerytherin, phycocyanin, allophycocyanin, o-phthaldehyde and fluorescamine, achemiluminescent labeling compound selected from the group comprising luminol, isoluminol, an aromatic acridinium ester, an imidazole, an acridinium salt and an oxalate ester, or a bioluminescent compound selected from the group comprising luciferin, luciferase and acquorin.

Examples of chromogenic substances include, but are not limited to, 5-Bromo-4-chloro-3-indolyl phosphate, 5-Bromo-6-chloro-3-indolyl phosphate p-toluidine, 3,3′-(3,3′-dimethoxy-4,4′-biphenylylene)-bis-2-(p-nitrophenyl)-5-phenyl-2- H-tetrazolium chloride, 5-Bromo-4-chloro-3-indolyl-β-D-galactopyranoside, 5-Bromo-6-chloro-3-indolyl-β-D-galactopyranoside, 5-Bromo-3-indolyl-β-D-galactopyranoside, 6-Bromo-2-naphthyl-β-D-galactopyranoside, 6-Chloro-3-indolyl-β-D-galactopyranoside, 6-Bromo-3-indolyl-β-D-galactopyranoside, 1-Methyl-3-indolyl-β-D-galactopyranoside, o-Nitrophenyl-β-D-galactopyranoside, p-Nitrophenyl-β-D-galactopyranoside, 3,4-cyclohexenoesculetin-β-D-galactoside, 8-hydroxychinoline-β-D-galactoside, 5-Bromo-4-chloro-3-indolyl-β-D-galactopyranoside, 5-Bromo-4-chloro-3-indolyl-β-D-glucopyranoside, 5-Bromo-4-chloro-3-indolyl-β-D-glucuronide, 5-Bromo-6-chloro-3-indolyl-β-D-glucuronide, 8-hydroxyquinoline-β-D-glucuronide, 2,2′-Azino-bis(3-ethylbenzothiazoline-6-sulfonic acid), 4-Chloro-1-naphthol, 3,3′-Diaminobenzidine tetrahydrochloride, o-Phenylenediamine, 3,3′,5,5′-Tetramethylbenzidine, 4-[2-(4-octanoyloxy-3,5-dimethoxyphenyl)-vinyl]-quinolinium-1-(propan-3-y-1-carboxylic-acid)-bromide, 5-Bromo-6-chloro-3-indolyl-caprylate, 5-bromo-4-chloro-3-indoxyl-myo-inositol-1-phosphate.

Examples of metal ions include, but are not limited to, chromium (III), manganese (II), iron (III), iron (II), cobalt (II), nickel (II), copper (II), neodymium (III), samarium(III), ytterbium (III), gadolinium (III), vanadium (II), terbium (III), dysprosium (III), holmium (III) and erbium (III), or a radioopaque material, such as barium, diatrizoate, ethiodized oil, gallium citrate, iocarmic acid, iocetamic acid, iodamide, iodipamide, iodoxamic acid, iogulamide, iohexol, iopamidol, iopanoic acid, ioprocemic acid, iosefamic acid, ioseric acid, iosulamide meglumine, iosemetic acid, iotasul, iotetric acid, iothalamic acid, iotroxic acid, ioxaglic acid, ioxotrizoic acid, ipodate, meglumine, metrizamide, metrizoate, propyliodone, and thallous chloride.

The invention also provides molecularly imprinted polymer platforms in which templated sites are formed for specific target molecules. In one embodiment, the polymer platforms can be provided wherein the templated sites have at least one reporter molecule bonded to a reactive group at the site. In one embodiment, the polymer platform comprises xerogels or aerogels. Methods to develop sensors for the detection of a wide variety of targets are described by Bright et al., in U.S Publication No. 2005/0227258, the portions of which that describe development of templated sites for specific target molecules, is incorporated herein by reference.

For example, the template is chosen such that it forms bonds with the polymer platform in excess of the number of those which will be formed by the bound target. The template can have at least one additional reactive group so as to be able to bind to the polymer matrix at an additional site relative to an intended target. In one embodiment, the number of reactive groups on the template which link it to the polymer platform, is at least one more than the number of reactive groups on the corresponding target. The removal of the template results in cavities within the polymer platform. Exposed at each templated site are reactive groups which are responsible for target recognition. However, as a consequence of the additional reactive group(s) mentioned above, when the template is cleaved from the cavity, the cavity bears one or more reactive groups in excess of the groups needed to bind the target. The extra group(s) is (are) used to bond with reporter molecules. Once the reporter molecule(s) is (are) bound at the templated site, the absorbance/luminescence from the target bound MIP can be measured and a change in UV, visible or IR absorbance/luminescence properties of the reporter (e.g., absorbance spectra, excitation and emission spectra, excited-state luminescence lifetime and/or luminescence polarization) indicates the presence of target at the templated site. The total change in absorbance/luminescence is generally proportional to the concentration of target molecule in the sample.

The MIPs of the invention can be utilized for diagnosing a disease condition associated with target organs, including, but not limited to, prostate, brain, myocardium, thyroid, lungs, liver, gallbladder, kidneys.

It is to be understood that application of the teachings of the present invention to a specific problem or environment will be within the capability of one having ordinary skill in the art in light of teachings contained herein. The present invention is more fully illustrated by the following non-limiting examples.

### EXAMPLES

### Example 1

### Molecular Imaging of Prostate Cancer

The principle of the invention is demonstrated by targeting and imaging solid tumors such as prostate cancer. This is accomplished herein for prostate cancer by providing a graded approach consisting of a) synthesis of MIPs of appropriate stealth for in vivo applications, i.e., particles that will avoid the reticuloendothelial system, b) in vitro testing of suitably derivatized particles in PSMA+LNCaP or PIP cells vs. PSMA-PC3 cells, c) quantitative, ex vivo biodistribution in SCID mice harboring a PSMA+ and PSMA− tumor in opposite flanks and d) in vivo imaging of SCID mice. Key to the entire approach is having amine- or carboxy-functionalized MIPs that can be derivatized for imaging, using a variety of possible agents, including those radiolabeled with ¹²⁵I, ^{99m}Tc and ¹⁸F as well as those tagged with fluorphores, particularly those that emit in the near-infrared region of the spectrum for tissue penetration. MIPs are also derivatized for stealth. The approach is iterative and can produce a variety of functionalized, target particles. Additionally, the length of the linker between the MIP and the “chelator” for the imaging moiety may be optimized for ideal in vivo pharmacokinetics.

Non-limiting examples of targets that MIPs of the invention described herein can be directed to include the CD 133 antigen (on cancer stem cells), the prostate-specific membrane antigen (PSMA), the avb3 integrin receptor, HER2/neu receptor, CXCR4 receptor, somatostatin receptor, Muc1, hepsin, and uPAR. Antibody or low molecular weight imaging agents exist for each of these targets, which have had varying degrees of success. PSMA and particularly avb3 integrin have the advantage of being within tumor neovasculature such that the MIPs would not need to transgress any barriers to gain access to target. Additional examples of targets include various tyrosine kinase receptors, and the epithelial cell adhesion molecule. Such target molecules are novel and highly desirable for cancer imaging.

MIPs conjugated to a radionuclides such as ¹²⁵I, ^{99m}Tc and ¹⁸F as well as those tagged with fluorphores attached and developed to bind specifically to epitopes of the CD133 antigen, PSMA), the avb3 integrin receptor, HER2/neu receptor, CXCR4 receptor, somatostatin receptor, Muc1, hepsin, and uPAR may be more effective radiopharmaceutical. A gamma camera can be used to take a picture of the prostate to determine if there is any tumor growth in the prostate. If the image is positive, a biopsy can be performed to determine the nature of the tumor. If the image is negative, no biopsy need be performed. This is a non-invasive step in determining the presence of prostate cancer tumor. This procedure could be expanded to provide diagnoses of the presence of many tumors wherein a specific antigen can be targeted by MIPs with a radionuclide attached.

### Example 2

### Molecular Imaging of Malignant Hematopoietic Cells

The principle of this invention is further demonstrated by the targeting and imaging of malignant leukocytes in vivo that is indicative of leukemias and lymphomas. White blood cells, or leukocytes are cells of the immune system involved in defending the body against both infectious disease and foreign materials. Five different and diverse types of leukocytes exist, but they are all produced and derived from a multipotent cell in the bone marrow known as a hematopoietic stem cell. Lymphoma is a cancer in the lymphatic cells of the immune system. Typically, lymphomas present as a solid tumor of lymphoid cells.

Leukemia and lymphoma cells have cell surface molecules that that can be targeted by MIPs derivatized with an imaging agent such as ^{99m}Tc, ¹¹¹In, ¹²³I, ²⁰¹Tl. A gamma ray camera can then take pictures of where these cells are located in vivo. The ability to distinguish malignant leukocytes from normal leukocytes and where they are located within a living body can have medical significance.

### Example 3

### Molecular Imaging of Internal Infections

The principle of this invention is further demonstrated by the targeting and imaging of microorganisms in vivo that cause internal infections. Imaging of internal infections can contributed significantly to the way researchers study bacterial pathogens and develop pre-clinical treatments to combat their ensuing infections in vivo. Not only does this approach allow disease profiles and drug efficacy studies to be conducted non-destructively in live animals over the entire course of the disease, but in many cases, it will enable investigators to observe disease profiles that could otherwise easily be missed using conventional methodologies.

The invention described above should be read in conjunction with the accompanying claims and drawings. The description of embodiments and examples enable one to practice various implementations of the invention and they are not intended to limit the invention to the preferred embodiment, but to serve as a particular example of the invention. Those skilled in the art will appreciate that they may readily use the conception and specific embodiments disclosed as a basis for modifying or designing other methods and systems for carrying out the same purposes of the present invention.

All references, including publications, patent applications, patents, and website content cited herein are hereby incorporated by reference to the same extent as if each reference were individually and specifically indicated to be incorporated by reference and was set forth in its entirety herein.

The use of the terms “a” and “an” and “the” and similar referents in the context of describing the invention are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein. The word “about,” when accompanying a numerical value, is to be construed as indicating a deviation of up to and inclusive of 10% from the stated numerical value. The use of any and all examples, or exemplary language (“e.g.” or “such as”) provided herein, is intended merely to better illuminate the invention and does not pose a limitation on the scope of the invention unless otherwise claimed. No language in the specification should be construed as indicating any non-claimed element as essential to the practice of the invention.

What is claimed is:

1. A biocompatible molecularly imprinted polymer (MIP), wherein the MIP is non-toxic, water soluble, small molecular weight, and degradable within a living body.

2. The MIP of claim 1, wherein the MIP is derivatized for stealth.

3. The MIP of claim 2, wherein said MIP comprises polyethylene glycol polymer or a derivative thereof.

4. The MIP of claim 1, wherein the MIP is functionalized to an amine or carboxyl group and is derivatized for imaging activity.

5. The MIP of claim 1, wherein the MIP is functionalized to an amine or carboxyl group and is derivatized for therapeutic activity.

6. The MIP of claim 1, wherein the MIP specifically binds a target macromolecule or portion thereof located within said living body.

7. The MIP of claim 6, wherein said macromolecule is a cancer antigen.

8. The MIP of claim 7, wherein said cancer antigen is associated with a tumor.

9. The MIP of claim 7, wherein said cancer antigen is associated with a malignant hematopoietic cell.

10. The MIP of claim 6, wherein said macromolecule is a part of a microorganism.

11. The MIP of claim 6, wherein said macromolecule is selected from a group consisting of proteins, glycoproteins, lipoproteins, peptidoglycans, peptides, polypeptides, polynucleotides and polysaccharides.

12. The MIP of claim 4, wherein said imaging agent is selected from a group consisting radionuclides, fluorophores, metalloproteins, iodine, metals, gadolinium, gold, and platinum.

13. The MIP of claim 5, wherein said therapeutic agent is selected from a group consisting of radionuclides, toxins, metals, gold, silver, and platinum.

14. The MIP of claim 6, wherein the MIP is injected, ingested, or inhaled into a living body.

15. A method of detection of a medical condition or treatment of a medical condition, wherein said condition comprises the presence of a macromolecule associated with that condition, said method comprising:
providing a MIP which specifically associates in vivo with said macromolecule, and
delivering said MIP to a patient,
wherein said MIP specifically binds said macromolecule or a portion thereof, said MIP is conjugated to comprise at least one imaging or at least one therapeutic agent, and said MIP is engineered for in-vivo stealth.

16. The method of claim 15, wherein said MIP is engineered for said increased stealth by attachment of polyethylene glycol to said MIP.

17. The method of claim 15, wherein said macromolecule is selected from a group consisting of proteins, glycoproteins, lipoproteins, peptidoglycans, peptides, polypeptides, polynucleotides and polysaccharides.

18. The method of claim 15, wherein said macromolecule is the CD 133 antigen, PSMA, avb3 integrin receptor, HER2/neu receptor, CXCR4 receptor, somatostatin receptor, Muc1, hepsin or uPAR.

19. The method of claim 15, wherein said macromolecule is a coat associated molecule of an infectious agent.

20. The method of claim 15, wherein said imaging agent is selected from a group consisting of radionuclides, fluorphores, metalloproteins, iodine and metals such as gadolinium, gold, and platinum.

21. The method of claim 15, wherein the therapeutic agent is selected from a group consisting of radionuclides, toxins, and metals such as gold, silver, and platinum.
